(19) United States
(12) Patent Application Publication
(10) Pub. No.: US 2009/0204157 A1
(43) Pub. Date: Aug. 13, 2009

(54) Locking Bone Plates with Controlled Locking Screw Misalignment

(76) Inventor: Alberto A. Fernandez Dell'Oca, Montevideo (UY)

Correspondence Address: Fay Kaplun & Marcin, LLP, 150 Broadway, suite 702, New York, NY, 10038, US

(21) Appl. No.: 12/294,292

(22) PCT Filed: Mar. 28, 2007
(86) PCT No.: PCT/US2007/007874

§ 371 Date: Mar. 30, 2009

Related U.S. Application Data
(60) Provisional application No. 60/786,390, filed on Mar. 28, 2006.

(51) Int. Cl. A61B 17/80; A61B 17/58

(52) U.S. Cl. 606/280; 606/99

(57) ABSTRACT

A bone plate system for fixing bone fractures includes a locking bone plate and a conically-shaped drilling sleeve. The bone plate has at least one threaded hole, and with the drilling sleeve screwed into the bone plate hole, a drill bit guided through the sleeve drills a hole into bone having a central axis angularly misaligned with the central axis of the bone plate hole. The angle between the drilled hole's central axis and the bone plate hole's central axis is equal to or less than the tolerance angle of the threads of the bone plate hole. This allows a bone plate screw to be imperfectly seated with respect to the bone plate hole and still screwed into the hole to lock the plate without jamming the screw head into the plate hole, while still allowing the screw to be screwed out if needed.

### FIELD OF THE INVENTION

The invention relates generally to implanted bone plate systems for fixing bone fractures. More particularly, the invention relates to locking bone plates with locking screws that are intentionally and controllably misaligned angularly through holes in the bone plate.

### BACKGROUND OF THE INVENTION

Since the early 20th century, bone plates and screws have been used for internal fixation of broken bones. As of the late 1980's, locking bone plates were developed. A locked bone plate uses a locking screw that has threads on an outer surface of its head that mates with corresponding threads in the bone plate hole. Because of the fixed relationship between locking screws and the bone plate, locking screws provide high resistance to shear or torsional forces. Thus, the main feature of such “locking bone plates” is a solid fixation between the plate and inserted screws. The advantages of locked plates—angular stability, less bone vascular damage, better infection resistance—became evident. Since then, use of locked plates has exploded, and they are now produced by different manufacturers.

Different systems have been developed to solidly lock the screw head to the plate hole. In most of these known devices, the locked screw has to be inserted at a predetermined angle. Should the surgeon insert the locking screw at a different angle, either the screw will not lock, or the screw will only lock provisionally, providing little or no angular stability and ultimately giving way under load. (Although there is an angular screw tolerance of some degrees, which can vary from system to system depending on the manufacturer, staying within that tolerance is difficult). An example of such a device is disclosed by Tepic in U.S. Pat. No. 5,151,103.

Other disadvantages of known locking systems and insertion procedures include jamming of the screw head in the plate hole (considered by some as cold welding), which has often become a nightmare when the surgeon needs to remove the locked screw. Sometimes the surgeon was forced to cut apart the plate within the patient in order to remove the locked screw. This can result in serious tissue damage and put the internal fixation at considerable risk.

In order to remedy these disadvantages, various changes to known bone plate systems were made, including changes to the design of the plate hole and/or screw head, the precision of the insertion technique of the screw, the amount of insertion torque used, and the type of metals used to form the screws and plates, among others. However, these changes have failed to provide an adequate solution to the disadvantages described above.

Therefore, a need still exists to provide devices, systems, and methods having the advantages of locked plates, while preventing excessive locking and allowing reliable and safe removal of the locking screw, if needed.

### SUMMARY OF THE INVENTION

It is an object of the invention to provide a locking bone plate device and system that permits safe and reliable removal of the locking screw, should it be needed, while maintaining the advantages of locking plates, including angular stability, less bone vascular damage, and better infection resistance, among others.

The invention addresses the main reason why locking screws are often jammed in the plate: the three-dimensional geometrical problem of a too perfect match among the threads in the plate hole and on the locking screw.

Generally, the better the surgical technique, the more perfect the position of the locking screw with respect to the plate hole, the more stable the assembly—but, on the other hand, the more difficult it may become to remove the screw afterwards, should it be required, because of the likelihood of jamming.

The invention therefore includes an advantageously conical drilling sleeve that guides a drill bit though a bone plate to drill a hole in bone. The drilled hole has an axis which differs slightly (i.e., does not coincide with) the axis of the bone plate hole. The drilling sleeve engages the locking bone plate and guides a drill bit to drill a hole for a locking screw.

The conical drilling sleeve of the invention guides the drill bit so as to drill a bone hole of which the axis does not exactly coincide or angularly align with the axis of the plate hole. However, even though the axes differ slightly, the axis of the drilled bone hole is still at or within the tolerance angle required to maintain good mechanical performance of locked implants.

A locking screw is then inserted into the drilled bone hole, the axis of the locking screw thus being slightly different than the hole axis, yet this difference is still within the tolerance required to maintain proper mechanical performance of the screw-plate coupling. This insertion technique, referred to as “controlled misalignment,” provides satisfactory mechanical performance while avoiding the jamming between the plate hole and the locking screw. This permits the surgeon to reliably and safely remove the locked screw, when necessary.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 is a perspective view of a known bone plate system;

FIG. 2 is a perspective view of the known system of FIG. 1 showing a conventional cylindrical sleeve threaded into the threaded bone plate hole with a drill bit having drilled a hole into bone;

FIG. 3 is a cross-sectional view of the known system of FIG. 1 after removal of the drill bit and conventional sleeve showing the drilled bone hole having a central axis that coincides with the central axis of the plate hole;

FIG. 4 is a cross-section of the known system of FIG. 1 showing a bone plate screw perfectly screwed into the threaded bone plate hole;

FIG. 5 is a perspective view of a bone plate system according to the invention;

FIG. 6 is a perspective view of the system of FIG. 5 showing the conical sleeve threaded into the threaded bone plate hole with a drill bit having drilled a hole into bone;

FIG. 7 is a cross-sectional view of the system of FIG. 5 after removal of the drill bit and conical sleeve showing the bone hole drilled in the controlled misalignment position; and

FIG. 8 is a cross-sectional view of the system of FIG. 5 showing a bone plate screw imperfectly screwed into the threaded bone plate hole according to the invention.

### DETAILED DESCRIPTION OF THE INVENTION

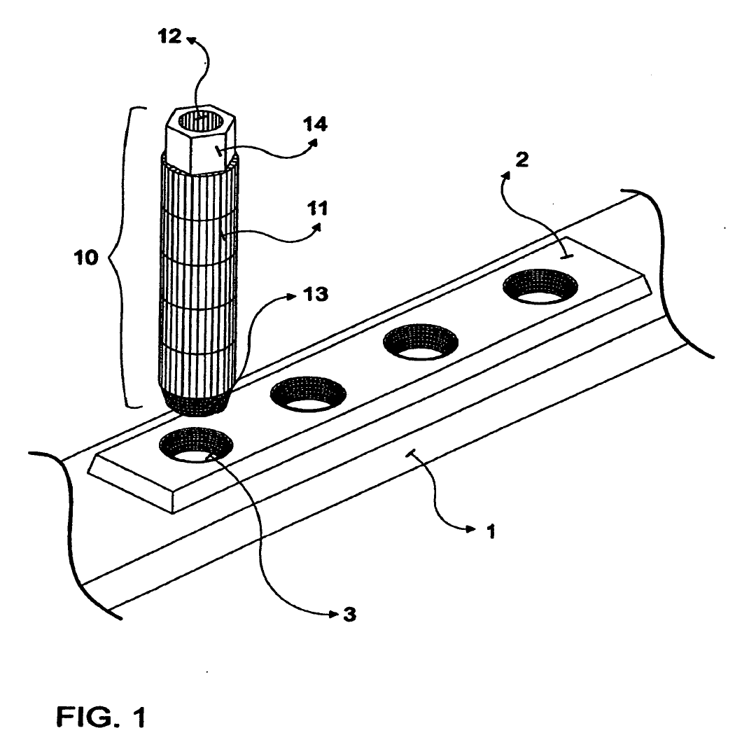

FIGS. 1-4 illustrate a conventional locking bone plate system that includes a conventional locking plate 2, a conventional cylindrical drilling sleeve 10, and a conventional locking screw 4.

Conventional bone plate 2 typically has at least two threaded holes, which in this example are conical threaded holes 3, designed to receive locking screws 4. Locking screws 4 have a threaded head 5, which in this example is conically shaped, and a threaded shaft 6 extending from head 5. The external threads on threaded head 5 mate with and preferably perfectly match the female threads in each of plate holes 3.

Conventional cylindrical sleeve 10 has a threaded head 13 that also mates with and preferably perfectly matches the female threads in each of plate holes 3.

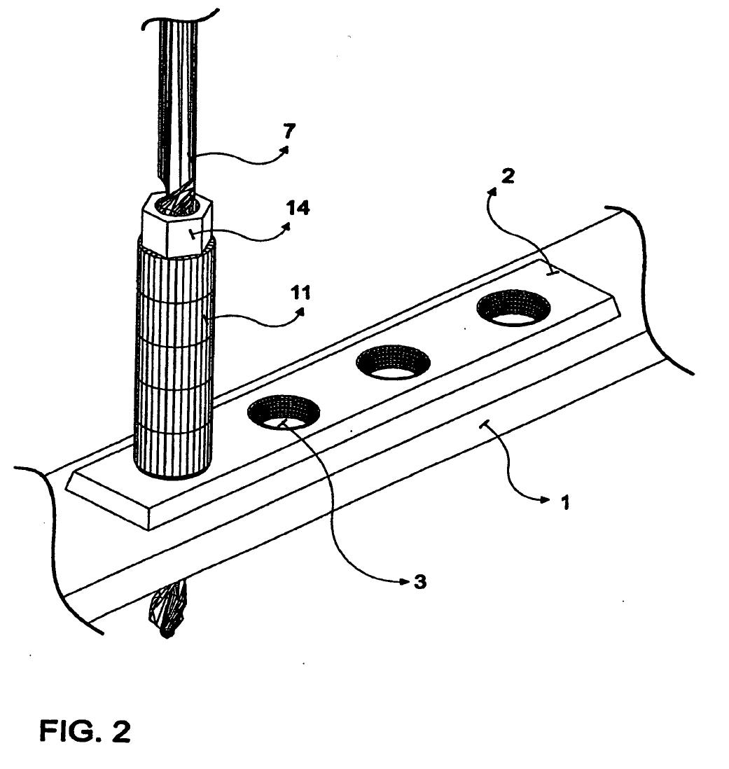

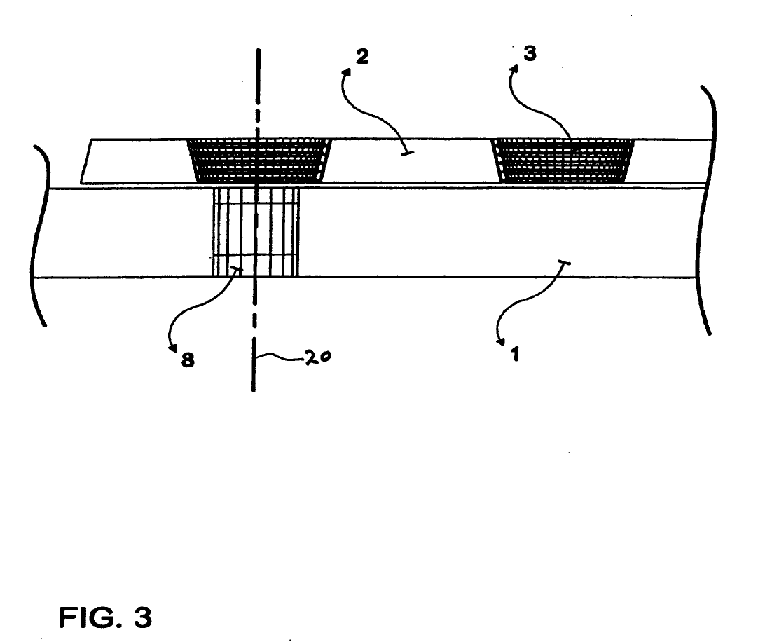

Once the conventional cylindrical sleeve 10 is properly positioned and screwed into one of plate holes 3, as shown in FIG. 2, bore 12 in cylindrical body 11 of conventional sleeve 10 is operative to guide drill bit 7 there through to drill a hole 8 in bone 1. Hole 8 will have the same central axis 20 as plate hole 3, as shown in FIG. 3.

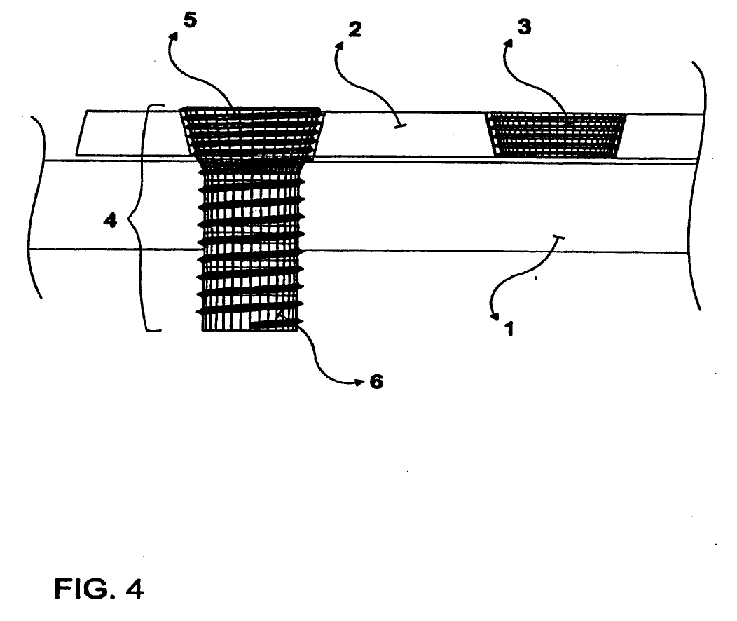

FIG. 4 shows the so-called “perfect position and orientation” of locking screw 4 screwed into plate hole 3 and drilled hole 8, with the central axis of the locking screw coincident with the central axes of the plate hole and drilled hole. In other words, the thread on head 5 of locking screw 4 is offset from and exactly parallel to the thread of plate hole 3, resulting in the “perfect” engagement of the threads in the head of screw 5 with the threads of plate hole 3.

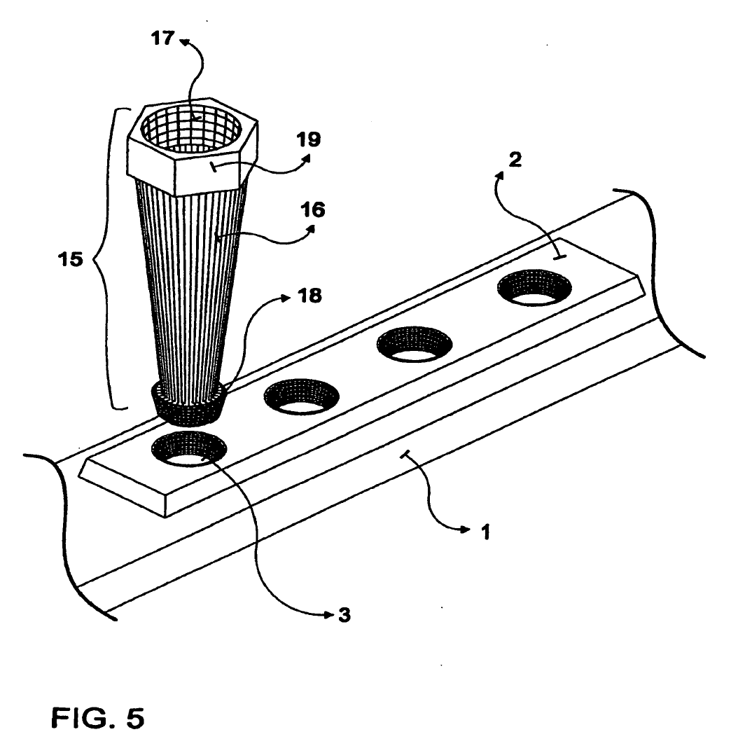

FIGS. 5 to 8 refer to a preferred embodiment of the bone plate system of the invention, which includes a locking bone plate 2 and a conical drilling sleeve 15. Conical drilling sleeve 15 engages locking plate 2 to guide a drill bit 7 there through for drilling a bone hole 9 to receive a locking screw 4.

In particular, locking plate 2, which may be a conventional locking bone plate, preferably has at least two threaded holes, which in this embodiment are conical threaded holes 3, designed to receive locking bone screws 4. Locking bone screws 4 have a threaded head 5, which in this embodiment is conically shaped to engage the female thread of any one of plate holes 3. Locking bone screws 4 also have a threaded shaft 6 extending from head 5 for engaging bone.

As shown in FIG. 5, conical drilling sleeve 15 has a threaded head 18 at its front end and a hexagonal head 19 at its rear end. Hexagonal head 19 is designed to accommodate a wrench, and threaded head 18 mates with and preferably perfectly engages the female thread at each plate hole 3. The cone angle is such that, when conical sleeve 15 is engaged in a plate hole 3, the angle between the inner cone wall in bore 17 and the central axis of plate hole 3 is equal to or less than the tolerance angle (which may differ between different manufacturers of locked bone plates and screws). The tolerance angle is the maximum angle as measured from the central axis of the threaded plate hole at which a bone plate screw can be positioned at the plate hole and the threaded head of the bone plate screw still screwed into and out of the threaded hole.

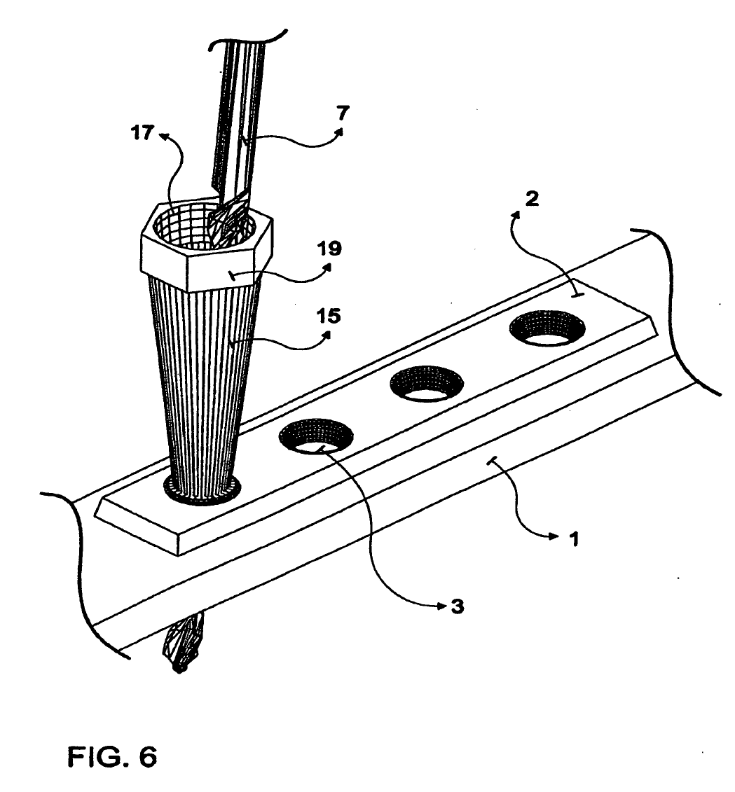

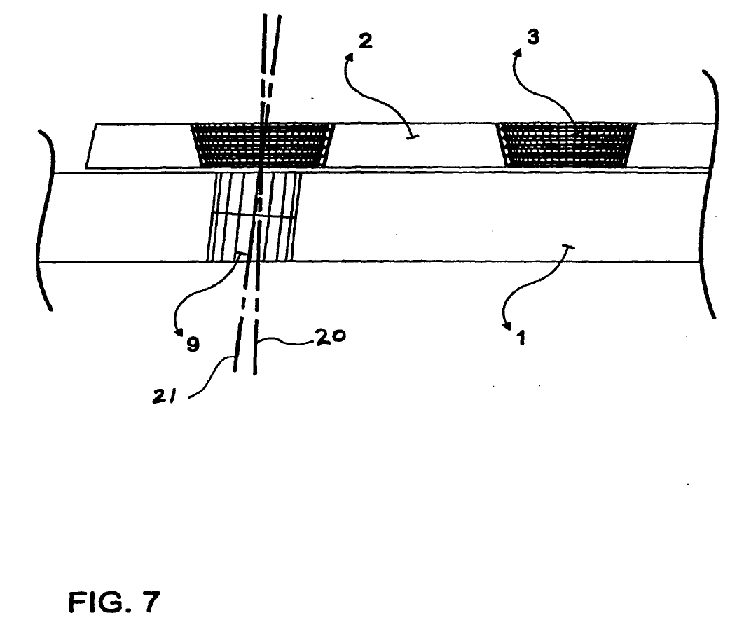

Once the conical sleeve 15 is perfectly positioned in a plate hole 3, drill bit 7 is inserted through conical bore 17 of sleeve 15 such that the drill bit preferably contacts and is guided by the inner wall of conical bore 17, as shown in FIG. 6. This causes drill bit 7, guided by the inner wall of conical sleeve 15, to drill a hole 9 in bone 1 of which the hole's axis 21 will differ slightly from axis 20 of plate hole 3, as shown in FIG. 7. That is, axis 21 is angularly misaligned with axis 20. The difference between axis 21 of bone hole 9 (and thus the axis of locked screw 4 after insertion) and axis 20 of plate hole 3 is no more than, and preferably within, the tolerance angle, which assures satisfactory mechanical performance of the screw-plate coupling.

Locking screw 4 may then be imperfectly seated at a threaded hole 3 by placing shaft 6 in drilled hole 9. In other words, locking screw 4 is imperfectly seated at a threaded hole 3 when the thread on head 5 is offset from and only substantially parallel to the thread of threaded hole 3. Locking screw 4 may then be screwed into threaded hole 3 and drilled hole 9 by substantially engaging the thread on head 5 with the thread of threaded hole 3.

Therefore, positioning shaft 6 of bone screw 4 in drilled hole 9 ensures that the tolerance angle will not be exceeded when screwing screw 4 into a plate hole 3, thus avoiding the possibility of jamming the screw head into the plate and/or damaging the threads.

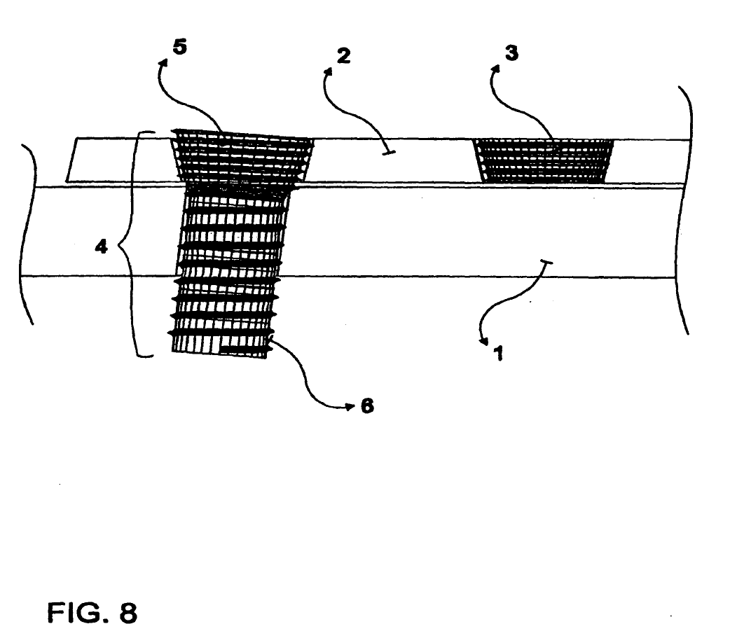

FIG. 8 shows locking screw 4 at its final position inside bone hole 9 with its axis slightly different (angularly misaligned) from the axis of plate hole 3.

The invention has been described in connection with the preferred embodiments. These embodiments, however, are merely for example and the invention is not restricted to them. It will be understood by those skilled in the art that other variations and modifications can be easily made within the scope of the invention as defined by the appended claims. Therefore, the invention is only intended to be limited by the following claims.

I claim:

1. A bone plate system comprising:
a bone plate having an upper surface, a lower surface, and a threaded hole extending through the upper and lower surfaces, the threaded hole having a central axis; and
a drilling sleeve comprising an elongated body having a conically-shaped bore extending longitudinally there through and forming an inside wall in the body, the body having an end at the narrowest portion of the bore configured to attach to the threaded hole; wherein:
with the drilling sleeve attached to the threaded hole, the inside wall forms an angle with the central axis of the threaded hole at which a bone plate screw having a head with a corresponding external thread thereon can be positioned and screwed into and out of the threaded hole without jamming or damaging any threads.

2. The system of claim 1 wherein the end of the body of the drilling sleeve at the narrowest portion of the bore has an external thread thereon that mates with the thread of the threaded hole.

3. The system of claim 1 further comprising a bone plate screw having a head with an external thread thereon that mates with the thread of the threaded hole, the bone plate screw further comprising a threaded shank extending from the head.

4. The system of claim 3 wherein the bone plate screw has a central axis angularly misaligned with the central axis of the threaded hole when the head of the bone plate screw is screwed into the threaded hole.

5. The system of claim 3 wherein the head of the bone plate screw is conically shaped.

6. The system of claim 1 wherein the threaded hole is conically shaped.

7. The system of claim 1 wherein the central axis of the threaded hole is orthogonal to the upper surface.

8. The system of claim 1 further comprising a plurality of threaded holes extending through the upper and lower surfaces.

9. A bone plate system comprising:
a bone plate having an upper surface, a lower surface, and a threaded hole extending through the upper and lower surfaces, the threaded hole having a central axis, the thread of the threaded hole having a tolerance angle at or within which a bone plate screw having a head with a corresponding external thread thereon can be imperfectly seated at the threaded hole and screwed into and out of the threaded hole, the bone plate screw having a central axis that coincides with the central axis of the threaded hole when perfectly seated and screwed into the threaded hole; and
a drilling sleeve comprising an elongated body having a conically-shaped bore extending longitudinally there through, the bore having an inner wall in the body, the body having an end at the narrowest portion of the bore, the end having an external thread thereon that mates with the thread of the threaded hole; wherein:
with the sleeve screwed into the threaded hole, the inner wall forms an angle with the central axis of the threaded hole that is equal to or less than the tolerance angle.

10. The system of claim 9 further comprising a bone plate screw having a head with a corresponding external thread thereon that mates with the thread of the threaded hole, the bone plate screw further comprising a threaded shaft extending from the head.

11. The system of claim 10 wherein the bone plate screw has a central axis angularly misaligned with the central axis of the threaded hole when head of the bone plate screw is screwed into the threaded hole.

12. The system of claim 10 wherein the head of the bone plate screw is conically shaped.

13. The system of claim 9 wherein the threaded hole is conically shaped.

14. The system of claim 9 wherein the central axis of the threaded hole is orthogonal to the upper surface.

15. The system of claim 9 wherein the bone plate screw is perfectly seated at the threaded hole when the thread of the bone plate screw is parallel to and offset from the thread of the threaded hole.

16. The system of claim 9 wherein the bone plate screw is imperfectly seated at the threaded hole when the thread of the bone plate screw is offset from and only substantially parallel to the thread of the threaded hole.

17. The system of claim 9 further comprising a plurality of threaded holes extending through the upper and lower surfaces.

18. A method of using a bone plate system comprising:
attaching a drilling sleeve to a threaded hole of a bone plate;
inserting a drill bit through a conically-shaped bore in the drilling sleeve;
drilling a hole with the drill bit guided by an inner wall of the conically-shaped bore such that a central axis of the drilled hole is angularly misaligned with a central axis of the threaded bone plate hole, the angular misalignment less than or equal to the tolerance angle of the thread of the threaded hole at which mechanical performance of a bone plate screw screwed into a bone plate hole is still satisfactory;
positioning the shaft of a bone plate screw through the bone plate hole and into the drilled hole; and
screwing the bone plate screw into the drilled hole until threads on a head of the bone plate screw substantially engage the threads of the threaded hole.

19. The method of claim 1 wherein the attaching a drilling sleeve comprises screwing the end of the drilling sleeve at the narrowest portion of the bore into a threaded hole of the bone plate, the end of the drilling sleeve having an external thread thereon that mates with the thread of the threaded hole.

20. The method of claim 1 further comprising unscrewing the bone plate screw out of the drilled hole and the threaded bone plate hole.
